(19) United States
(12) Patent Application Publication
(10) Pub. No.: US 2011/0178142 A1
(43) Pub. Date: Jul. 21, 2011

(54) PHARMACEUTICAL COMPOSITION FOR THERAPEUTIC OR PROPHYLACTIC TREATMENT OF BACTERIAL INFECTIONS AND ASSOCIATED DISEASES

(75) Inventors: Avadhesha Surolia, New Delhi (IN); Tushar Beuria Kant, Mumbai (IN); Panda Dulal, Mumbai (IN)

(73) Assignee: National Institute of Immunology, New Delhi (IN)

(21) Appl. No.: 13/122,074

(22) PCT Filed: Oct. 13, 2009
(86) PCT No.: PCT/IN2009/000573

§ 371 Date: Mar. 31, 2011

(30) Foreign Application Priority Data
Oct. 13, 2008 (IN) 2345/DEL/2008

(51) Int. Cl. A61K 31/427; C07D 409/02; A61P 31/04

(52) U.S. Cl. 514/365; 548/146

(57) ABSTRACT

The present invention provides a pharmaceutical composition comprising 3-{5-[4-Oxo-2-thioxo-3-(3-trifluoromethyl-phenyl)-thiazolidin-5-ylidenemethyl]-furan-2-yl}-benzoic acid or a pharmaceutically acceptable salt thereof and pharmaceutically acceptable excipients for therapeutic or prophylactic treatment of bacterial infections and diseases associated thereof.

### TECHNICAL FIELD

The present invention relates to a pharmaceutical composition comprising 3-{5-[4-Oxo-2-thioxo-3-(3-trifluoromethyl-phenyl)-thiazolidin-5 -ylidenemethyl]-furan-2-yl}-benzoic acid or a pharmaceutically acceptable salt thereof and pharmaceutically acceptable excipients for therapeutic or prophylactic treatment of bacterial infections and associated diseases.

### BACKGROUND OF THE INVENTION

Bacterial diseases are among the leading causes of human deaths and the most important health hazards in the developing parts of the world. The development of resistance to the existing drugs is also making the treatment ineffective. Thus, it is necessary to develop new antibacterial drugs with novel mechanism of action. Recent studies have shown that FtsZ plays an essential role in bacterial cell division.

FtsZ is a protein encoded by the FtsZ gene that assembles into a ring at the future site of the septum of bacterial cell division. FtsZ, named after “Filamenting temperature-sensitive mutant Z”, is a prokaryotic homologue to the eukaryotic cytoskeleton protein tubulin. FtsZ is highly conserved in bacteria and the perturbation of FtsZ functions has been shown to induce deleterious effects in bacteria (Baumann P. and Jackson S P. (1996) An archaebacterial homologue of the essential eubacterial cell division protein FtsZ. Proceedings of the National Academy of Sciences of the United States of America, 93, 6726-6730; Margolin W, Wang R. and Kumar M.(1996) Isolation of an FtsZ homolog from the archaebacterial Halobacterium salinarium: implications from the evolution of FtsZ and tubulin. The Journal of Bacteriology.178, 1320-1327). Though FtsZ has low sequence similarities with tubulin, both proteins share similar structural folds and identical GTP binding motifs (Erickson H P (1995) FtsZ, a prokaryotic homolog of tubulin? Cell. 80, 367-70; Erickson, H P (1998) Atomic structures of tubulin and FtsZ. Trends in Cell Biology. 8, 133-7; Lowe J and Amos L A. (1998) Nature. 391, 203-6; Nogales E, Wolf S G. and Downing, K H., (1998) Structure of the alpha beta tubulin dimmer by electron crystallography. Nature. 391, 199-203; Addinall S G. and Holland B (2002) The tubulin ancestors, FtsZ, draughtsman, designer and driving force for bacterial cytokinesis. Journal of Molecular Biology. 381, 219-36; Nogales E, Downing K H., Amos L A and Lowe J. (1998) Nature Structural Biology. 5, 452-8).

U.S. Pat. No. 4,352,929 discloses rhodanines to have anti-bacterial activity. U.S. Patent application No. 2002/0052396 discloses: 3-{5-[4-Oxo-2-thioxo-3-(3-trifluoromethyl-phenyla)-thiazolidin-5-ylidenemethyl]-furan-2-yl}-benzoic acid by the claimed Markush formula covering anti-viral compounds.

In bacteria, beta-hydroxyacyl-acyl carrier protein (ACP) dehydratase (FabZ) is a potent enzyme in fatty acid biosynthesis and catalyzes the dehydration of beta-hydroxyacyl-ACP to trans-2-acyl-ACP. It provides useful information in better understanding the FabZ of H. pylori strain and further supply possible hints in the discovery of anti-bacterial compounds using HpFabZ as target (Biochemical and Biophysical Research Communications. 2005 Aug 12;333(4),1078-86).

U.S. 2008/0051445 discloses 2-thioxothiazolodin-4-one compounds and compositions as antimicrobial and antimalarial agents targeting enoyl-ACP reductase of type II fatty acid synthesis pathway and other cell growth pathways.

Thus, there is a need to develop new antibacterial drugs with novel mechanism of action.

### SUMMARY OF THE INVENTION

One aspect of the present invention provides a pharmaceutical composition comprising an effective amount of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof and parmaceutically acceptable excipients, wherein the composition has antibacterial activity.

Another aspect of the present invention provides a method of treatment of bacterial infections and associated diseases therewith, wherein the method comprises administering a composition comprising an effective amount of 3-{5-[4-Oxo-2-thioxo-3-(3-trifluoromethyl-phenyl)-thiazolidin-5-ylidenemethyl]-furan-2-yl}benzoic acid or pharmaceutically acceptable salt thereof and pharmaceutically acceptable excipients.

Yet another aspect of the present invention provides use of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof for preparation of a medicament useful for the treatment of bacterial infections and associated diseases therewith.

Further aspect of the present invention provides use of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof in an effective amount ranging from 1 μM to 5 μM for preparation of a medicament useful for the treatment of bacterial infections and associated diseases therewith.

### BRIEF DESCRIPTION OF THE ACCOMPANYING DRAWINGS

FIG. 1 illustrates the rate and extent of FtsZ polymerization by compound of formula I at different concentration Shown are assembly reaction in the absence (•) and presence of 10 μM (♦), 25 μM (◯) and 50 μM (▴) of compound of formula I

FIG. 2 illustrates the increase in sedimentable polymer mass of FtsZ by compound of formula I at different concentrations.

FIG. 3 shows Electron Micrographs of FtsZ polymers using Transmission Electron Microscope after incubation with different concentrations of compound I and the control.

FIG. 4 illustrates the effects of compound of formula I on the GTPase activity of FtsZ.
A) illustrates the phosphate released per FtsZ per min in the absence and presence of 10 μM, 25 μM and 50 μM of compound of formula I at 5 min (), 15 min (▪) and 30 min (▴).
B) illustrates the total phosphate released in the absence () and presence of 25 μM (▴) of compound of formula I at different time points.

FIG. 5 illustrates the binding of compound of formula I with FtsZ.
A) illustrates the tryptophan emission intensity at 340 nm after incubation of FtsZ (Y371W) (1 μM) with different concentrations of compound of formula I.
B) illustrates the double reciprocal plot which is used to determine the dissociation constant.

FIG. 6 illustrates the Circular Dichroism (CD) measurements after incubation of FtsZ with different concentrations of compound of formula I.

FIG. 7 illustrates the calculation of minimum inhibitory concentration (MIC) of Bacillus subtilis.

FIG. 8 shows the induced filamentation in Bacillus subtilis.
A) Bacillus subtilis 168 as visualized under DIC microscope in control and after incubation with the compound of formula I.
B) illustrates the average length of the Bacillus subtilis 168 in control and 1 μM concentration of compound of formula I.

FIG. 9 shows the unperturbed inner membrane of Bacillus Subtilis in control and presence of compound of formula I as viewed under microscope with a 60X objective.

FIG. 10 shows immuno-stained Bacillus Subtilis as viewed under microscope with a 60X objective.

### DETAILED DESCRIPTION OF THE INVENTION

The present invention provides a pharmaceutical composition comprising 3-{5-[4-Oxo-2-thioxo-3-(3-trifluoromethyl-phenyl)-thiazolidin-5-ylidenemethyl]-furan-2-yl}-benzoic acid or a pharmaceutically acceptable salt thereof and pharmaceutically acceptable excipients for therapeutic or prophylactic treatment of bacterial infections and diseases associated therewith.

The present invention relates to a potential antibacterial agent using the assembly of FtsZ (filamenting temperature-sensitive mutant Z) monomers as the target for screening of a group of compounds of diverse chemical structures. The effects of 81 compounds were examined on the assembly of FtsZ monomers. Out of these 81 compounds, 3-{5-[4-Oxo-2-thioxo-3-(3-trifluoromethyl-phenyl)-thiazolidin-5-ylidenemethy]-furan-2-yl}-benzoic acid, hereafter referred as the compound was found to promote FtsZ assembly in vitro and potently inhibited bacterial proliferation.

The antibacterial mechanism of action of the compound is different from the previously known FtsZ assembly inhibitors like sanguinarine, totarol, curcumin and viriditoxin (Beuria, T K., Shah J H., Santra M K., Kumar V and Panda D. (2005) Sanguinarine blocks cytokinesis in bacteria by inhibiting FtsZ assembly and bundling. Biochemistry. 44, 16584-93; Jaiswal R., Beuria T K, Mohan R, Mahajan S K and Panda D. (2007) Totarol inhibits bacterial cytokinesis by perturbing the assembly dynamics of FtsZ. Biochemistry. 46, 4211-20; Wang J, Galgoci A, Kodali S, Herath K B, Jayasuriya H, Dorso K, Vicente F, Gonzalez A, Cully d, Bramhill D and Singh S. (2003) Discovery of a small molecule that inhibits cell division by blocking FtsZ, novel therapeutic target of antibiotics. The Journal of Biological Chemistry. 278, 44424-8; Rai D, Singh J K, Roy N and Panda D (2008) Curcumin inhibits FtsZ assembly: an attractive mechanism for its antibacterial activity. Biochemical Journal. 410, 147-155). The compound inhibits bacterial cytokinesis by enhancing FtsZ assembly and stability of the polymers thus potently inhibiting the bacterial proliferation.

The compound, one of the rhodanines, acts by interfering with the dynamics of Z-ring by enhancing of FtsZ assembly and the polymeric mass of FtsZ, causing bundling of FtsZ protofilaments. It prevents the dilution-induced disassembly of FtsZ protofilaments and decreases the GTPase activity in vitro. It was found that compound 3-{5-[4-Oxo-2-thioxo-3-(3-trifluoromethyl-phenyl)-thiazolidin-5-ylidenemethyl]-furan-2-yl}-benzoic acid inhibits bacterial cytokinesis by enhancing FtsZ assembly and/or the bundling of FtsZ protofilaments.

Another embodiment of the present invention provides identification of exact target for the action of rhodanines which is through FabZ protein needed for cytokinesis, a key event required for cell division of bacteria. Also, it is demonstrated that (Example 9) they act by interfering with the dynamics of Z-ring by enhancing of FtsZ assembly.

Yet another embodiment of the present invention provides that sedimentable polymeric mass of FtsZ increases by the action of 3-{5-[4-Oxo-2-thioxo-3-(3-trifluoromethyl-phenyl)-thiazolidin-5-ylidenemethy]-furan-2-yl}-benzoic acid, causes bundling of FtsZ protofilaments, prevents dilution-induced disassembly of FtsZ protofilaments and decreases the GTPase activity in vitro. High concentration of the compound induces aggregations of FtsZ monomers in vitro.

Yet another embodiment of the present invention provides minimum inhibitory concentration of 3-{5-[4-Oxo-2-thioxo-3-(3-trifluoromethyl-phenyl)-thiazolidin-5-ylidenemethy]-furan-2-yl}-benzoic acid required for inhibition of proliferation of Bacillus subtilis 168 cells. It was found that the minimum inhibitory concentration (MIC) is 3 μM. In the effective concentration range, the compound induced filamentation in bacteria and perturbed the formation of the cytokinetic Z-rings in bacteria. However, the compound neither perturbed the membrane structures nor did it affect the nucleoid segregation in Bacillus subtilis cells. The results suggested that the compound inhibited bacterial cytokinesis by perturbing the formation and functioning of the Z-ring by enhancing FtsZ assembly.

Another embodiment of the present invention provides a pharmaceutical composition comprising an effective amount of 3-{5-[4-Oxo-2-thioxo-3-(3-trifluoromethyl-phenyl)-thiazolidin-5-ylidenemethy]-furan-2-yl}-benzoic acid or a pharmaceutically acceptable salt thereof and pharmaceutically acceptable excipients, wherein the composition has antibacterial activity.

Further embodiment of the present invention provides a pharmaceutical composition comprising an effective amount of 3-{5-[4-Oxo-2-thioxo-3-(3-trifluoromethyl-phenyl)-thiazolidin-5-ylidenemethyl]-furan-2-yl}-benzoic acid, or a pharmaceutically acceptable salt thereof and pharmaceutically acceptable excipients, wherein the effective amount of said 3-{5-[4-Oxo-2-thioxo-3-(3-trifluoromethyl-phenyl)-thiazolidin-5-ylidenemethyl]-furan-2-yl}-benzoic acid or a pharmaceutically acceptable salt thereof is in the range of 1 μM to 5 μM.

Yet another embodiment of the present invention provides a pharmaceutical composition comprising an effective amount of 3-{5-[4-Oxo-2-thioxo-3-(3-trifluoromethyl-phenyl)-thiazolidin-5-ylidenemethyl]-furan-2-yl}-benzoic acid, or a pharmaceutically acceptable salt thereof and pharmaceutically acceptable excipients, wherein the composition comprises antibacterial agents known in the state of art.

Further embodiment of the present invention provides a pharmaceutical composition comprising an effective amount of 3-{5-[4-Oxo-2-thioxo-3-(3-trifluoromethyl-phenyl)-thiazolidin-5-ylidenemethy]-furan-2-yl}-benzoic acid, or a pharmaceutically acceptable salt thereof and pharmaceutically acceptable excipients, wherein the pharmaceutically acceptable excipients is an adjuvant, carrier or diluents or combinations thereof.

Still another embodiment of the present invention provides a pharmaceutical composition comprising an effective amount of 3-{5-[4-Oxo-2-thioxo-3-(3-trifluoromethyl-phenyl)-thiazolidin-5-ylidenemethyl]-furan-2-yl}-benzoic acid, or a pharmaceutically acceptable salt thereof and pharmaceutically acceptable excipients, wherein the composition optionally comprises supplementary active compounds selected from a group consisting of antibiotic, antiprotozoal agent, antifungal agent, antipathogen, and antiproliferative agent.

One embodiment of the present invention provides a pharmaceutical composition comprising an effective amount of 3-{5-[4-Oxo-2-thioxo-3-(3-trifluoromethyl-phenyl)-thiazolidin-5-ylidenemethyl]-furan-2-yl}-benzoic acid, or a pharmaceutically acceptable salt thereof and pharmaceutically acceptable excipients, wherein the composition is in the form of tablet, capsule, solution or powder.

Yet another embodiment of the present invention provides a pharmaceutical composition comprising an effective amount of 3-{5-[4-Oxo-2-thioxo-3-(3-trifluoromethyl-phenyl)-thiazolidin-5-ylidenemethyl]-furan-2-yl}-benzoic acid, or a pharmaceutically acceptable salt thereof and pharmaceutically acceptable excipients, wherein the mode of administration of the composition is intravenous, intramuscular or oral.

Still another embodiment of the present invention provides a method of treatment of bacterial infections and associated diseases, wherein said method comprises administering a composition comprising an effective amount of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof and pharmaceutically acceptable excipients.

Still another embodiment of the present invention provides a method of treatment of bacterial infections and associated diseases, wherein said method comprises administering a composition comprising an effective amount of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof and pharmaceutically acceptable excipients, the effective amount is in the range of 1 μM to 5 μM.

Another embodiment of the present invention provides use of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof for preparation of a medicament useful for the treatment of bacterial infections and associated diseases.

Further embodiment of the present invention provides use of3-{5-[4-Oxo-2-thioxo-3-(3-trifluoromethyl-phenyl)-thiazolidin-5-ylidenemethy]-furan-2-yl}-benzoic acid or pharmaceutically acceptable salt thereof in an effective amount ranging from 1 μM to 5 μM for preparation of a medicament useful for the treatment of bacterial infections and associated diseases.

One embodiment of the present invention provides a method of treatment of bacterial infections and associated diseases, wherein the antibacterial disease is caused by bacteria selected from a group consisting of E. coli or Bacillus subtilis.

While various embodiments and/or individual features of the present invention have been illustrated and described, it would be obvious to those skilled in the art that various other changes and modifications can be made without departing from the spirit and scope of the invention. As will be also be apparent to the skilled practitioner, all combinations of the embodiments and features taught in the foregoing invention are possible and can result in preferred executions of the present invention.

### EXAMPLES

The following examples are put forth so as to provide those of ordinary skill in the art with a complete invention and the description of how to make and use the present invention, and are not intended to limit the scope of what the inventors regard as their invention nor are they intended to represent that the experiments below are all and only experiments performed.

### Materials Used

Piperazine-N,N_-bis[2-ethanesulfonicacid](pipes),isopropyl-β-D-thiogalactopyranoside (IPTG), GTP, 6-diamidino-2-phenylindole (DAPI), and Cy3-conjugated goat anti-rabbit secondary antibody were obtained from Sigma Chemical Company. FM 4-64 was purchased from Molecular Probes, Eugene, OR. Primary polyclonal anti-FtsZ rabbit antibody was developed in rabbit against E. coli FtsZ by Bangalore Genei, India. All other chemicals used were of analytical grade.

### Example 1

### Purification of FtsZ Protein

FtsZ was over-expressed and purified from E. coli BL21 strain as described by Santa and Panda. (Detection of an intermediate during unfolding of bacterial cell division protein FtsZ: loss of functional properties precedes the global unfolding of FtsZ. (2003). The Journal of Biological Chemistry. 278, 21336-43). The recombinant protein is exactly same as the natural protein. For all the experimental procedures, the above expressed and purified recombinant FtsZ was used. The FtsZ concentration was measured by the Bradford method using BSA as a standard (Bradford M M. (1976) A rapid and sensitive method for the quantitation of microgram quantities of protein utilizing the principle of protein-dye binding. Analytical Biochemistry. 72, 248-54). The purified protein was frozen and stored at −80° C. Prior to use, FtsZ was thawed and centrifuged at 287,000 ×g for 30 min to remove any insoluble aggregates.

### Example 2

### Sedimentation Assay

FtsZ (12 μM) in 25 mM pipes buffer (pH 6.5) was incubated without or with different concentrations of 3-{5-[4-Oxo-2-thioxo-3-(3-trifluoromethyl-phenyl)-thiazolidin-5-ylidenemethyl]-furan-2-yl}-benzoic acid, hereafter referred as the compound for 10 min on ice. Then, 10 mM MgSO₄ and 1 mM GTP were added to the reaction mixtures and incubated for an additional 15 min at 37° C. Polymerized FtsZ was sedimented at 287,000×g for 30 mM, and the protein concentration in the supernatant was measured (Santra M K and Panda D. (2007) Acid-induced loss of functional properties of bacterial cell division protein FtsZ: evidence for an alternative conformation at acidic pH, Proteins. 67,177-88). The protein concentration in the pellet was calculated by subtracting the protein concentration in the supernatant from the total protein concentration.

FtsZ was polymerized in absence and presence of the compound at different concentrations and the polymers thus formed were pelleted by centrifugation. The compound enhanced the sedimentable polymeric mass of FtsZ (FIG. 2). For example, 50 μM concentration of the compound increased the polymeric mass of FtsZ by 25%.

### Example 3

### Light Scattering Assay

The effects of the compound on the kinetics of FtsZ assembly was monitored by 90° light scattering (Beuria T K, Krishnakumar S S, Sahar S, Singh N, Gupta K, Meshram M, Panda D. (2003) Glutamate-induced assembly of bacterial cell division protein FtsZ. The Journal of Biological Chemistry. 278, 3735-41). FtsZ (12 μM) in 25 mM pipes buffer (pH 6.5) was briefly incubated in the absence or presence of the compound for 15 min at room temperature and then, polymerized in the presence of 10 mM magnesium and 1 mM GTP. The light scattering intensity (500 nm) was monitored using a fluorescence spectrophotometer (JASCO FP 6500) at 37° C. (Beuria T K, Krishnakumar S S, Sahar S, Singh N, Gupta K, Meshram M, Panda D. (2003) Glutamate-induced assembly of bacterial cell division protein FtsZ. The Journal of Biological Chemistry. 278, 3735-41).

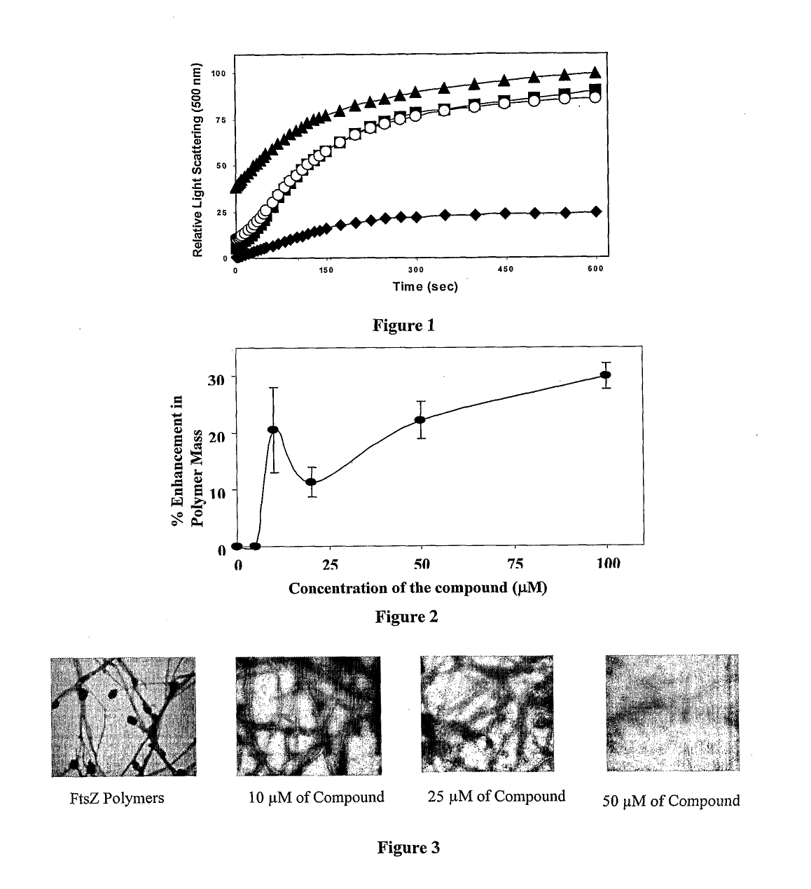

The effects of the compound on the assembly and bundling of FtsZ protofilaments were monitored using various complimentary techniques. First, the effects of the compound on the rate and extent of FtsZ assembly was monitored using 90° light scattering. The light scattering intensity was found to increase by ˜3 folds in the presence of 25 μM concentration, suggesting that it enhanced the assembly and/or the bundling of FtsZ protofilaments (FIG. 1). Shown are the assembly reaction in absence (♦) and presence of 10 μM (▪), 25 μM (◯) and 50 μM (▴) of the compound).

### Example 4

### Electron Microscopic Analysis of FtsZ Assembly

FtsZ (12 μM) in 25 mM pipes buffer (pH 6.5) was incubated without or with different concentrations of compound for 10 min on ice. Then, 10 mM MgSO4 and 1 mM GTP were added to the reaction mixtures and incubated for an additional 15 min at 37° C. Samples were transferred onto a formvar-carbon coated copper grid, stained with 2% uranyl acetate and observed using Transmission Electron Microscope (FEI TECHNAI G² 12) as described previously (Beuria T K, Santra M K and Panda D. (2005) Sanguinarine blocks cytokinesis in bacteria by inhibiting FtsZ assembly and bundling. Biochemistry. 44, 16584-93).

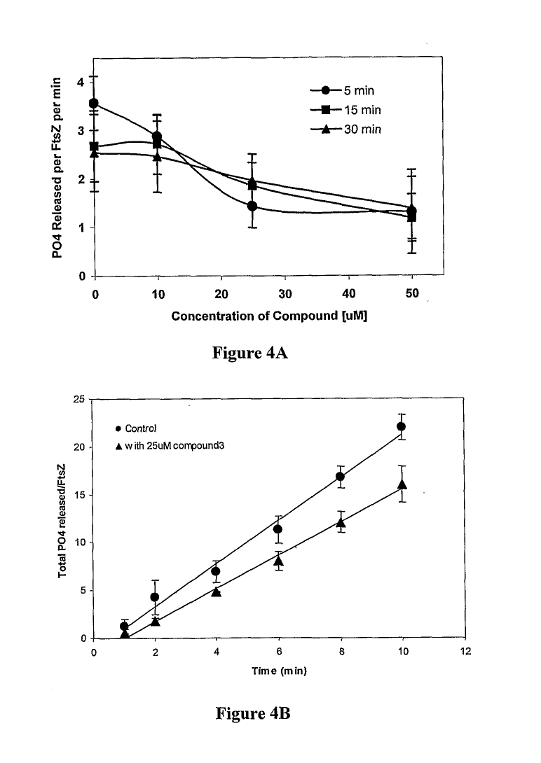

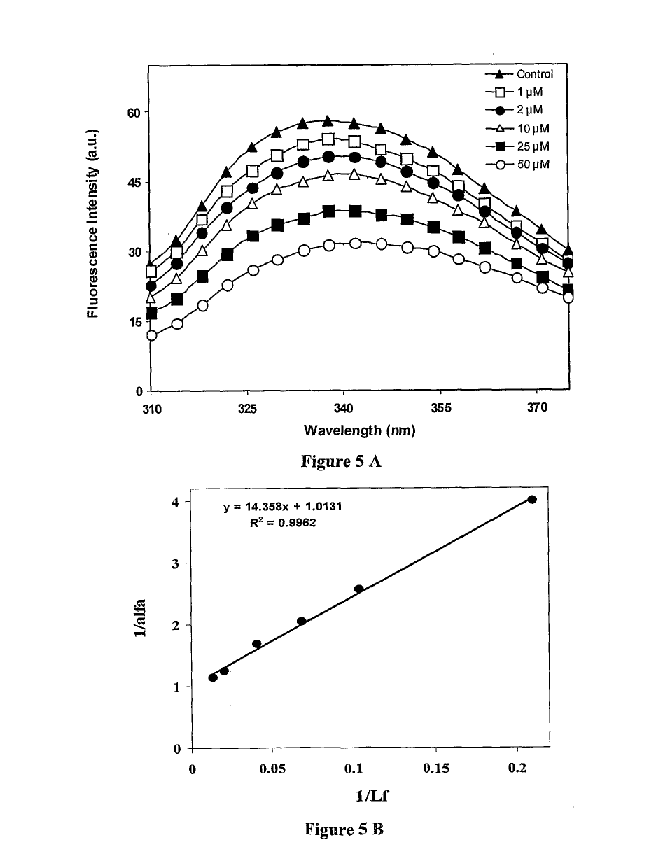

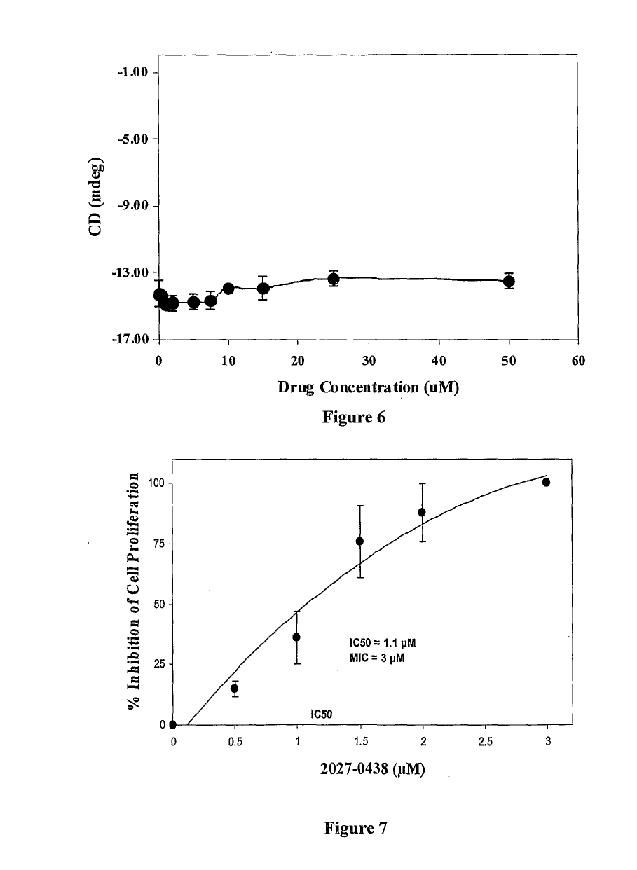

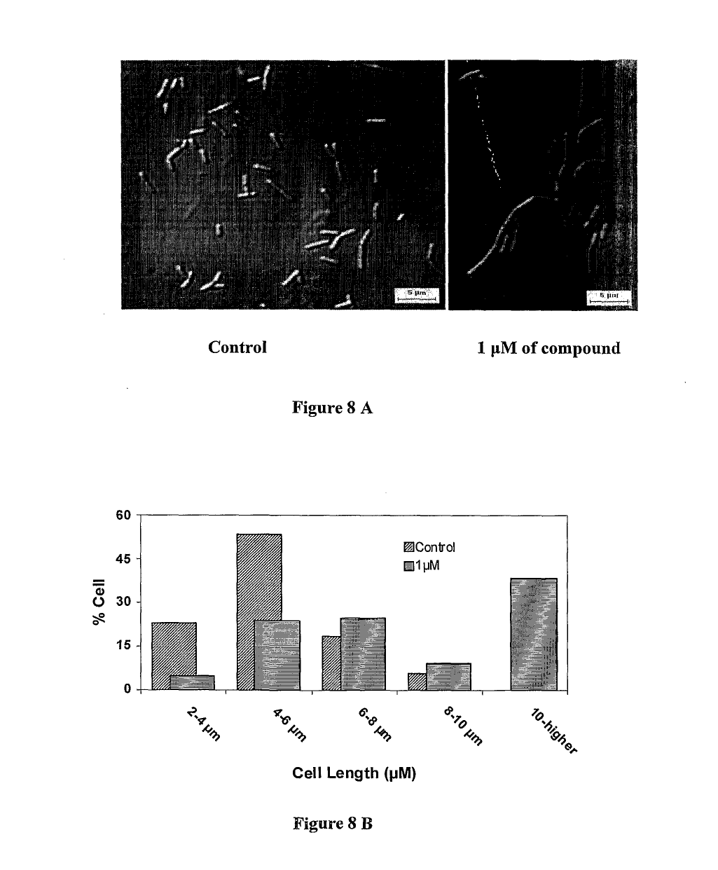

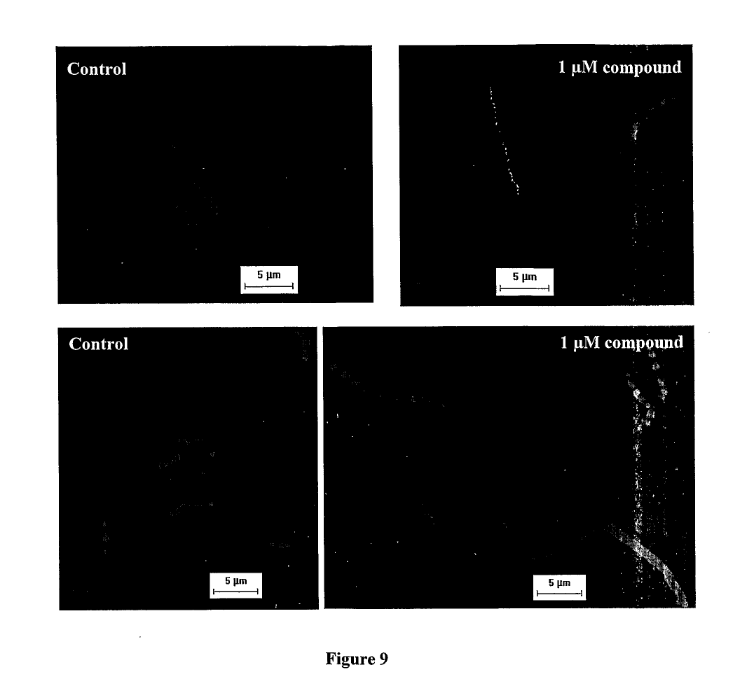

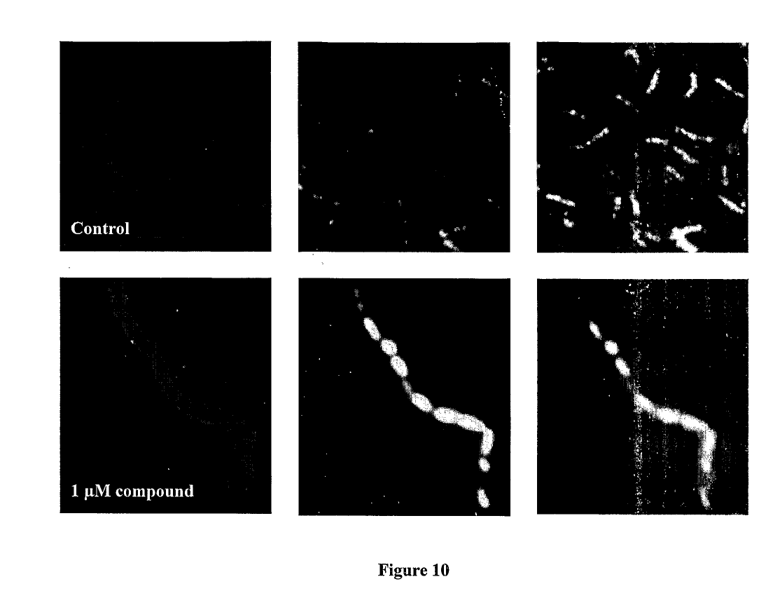

Electron microscopic analysis of the FtsZ polymers showed that the thickness of FtsZ bundles increased in the presence of the compound (FIG. 3). For example, the average thickness of the bundles were found to be 48±2.4, 64±3, and 66±4.3 nm in the absence and presence of compound at 10 μM and 25 μM concentrations respectively. The observed difference in the thickness of the FtsZ protofilament bundles in the absence and presence of 10 and 25 μM concentration is significant at a 99.9% confidence level (p<0.001). However, at 50 μM concentration, a large number of aggregates were observed and the average thickness of FtsZ bundles was found to be 46±3.5 nm.

### Example 5

### Effects of the Compound on Disassembly of Preformed FtsZ Filaments

FtsZ (30 μM) was polymerized in 25 mM pipes buffer in the presence of 5 mM MgCl₂, 1 mM GTP and 50 mM KCl. Preformed FtsZ polymers were diluted 20 fold in warm 25 mM pipes buffer in the absence and presence of different concentrations of Compound 1 and the mixtures were incubated for 5 min at 37° C. The polymers were then collected by centrifugation. The compound strongly prevented the dilution-induced disassembly of FtsZ protofilaments in a concentration dependent manner indicating that it stabilizes FtsZ protofilaments.

### Example 6

### Measurement of GTPase Activity

FtsZ (6 μM) in 25 mM pipes buffer (pH 6.5) was incubated with different concentrations of the compound for 30 min at 25° C. It was then polymerized in the presence of 10 mM magnesium and 1 mM GTP at 37° C. The GTP hydrolysis reaction was quenched at different time points by adding perchloric acid. Moles of inorganic phosphate released per mole of FtsZ were measured using the standard malachite green ammonium molybdate assay (Geladopoulos T P, Sotiroudis T G and Evangelopoulos A E. (1991). A malachite green colorimetric assay for protein phosphatase activity. Analytical Biochemistry.192,112-6;Beuria T K, Krishnakumar S S, Sahar S, Singh N, Gupta K, Meshram M and Panda D. (2003) Glutamate-induced assembly of bacterial cell division protein FtsZ. The Journal of Biological Chemistry. 278, 3735-41).

The rate and the extent of GTPase activity of FtsZ was found to decrease in the presence of the compound. (The rate of GTP hydrolysis at 5 min was found to be 3.6, 2.9, 1.5, and 1.3 (phosphate release per FtsZ per min) in the absence and presence of compound at 10, 25 and 50 μM concentration, respectively.

### Example 7

Interaction of the Compound with FtsZ

FtsZ (Y371 W) (1 μM) was incubated without or with different concentrations of the compound at 25° C. for 30 min. When excited at 290 nm, FtsZ (Y371W) displayed a typical emission spectrum with a maximum at 340 nm (Beuria T K, Santra M K and Panda D. (2005) Sanguinarine blocks cytokinesis in bacteria by inhibiting FtsZ assembly and bundling. Biochemistry. 44, 16584-93). The compound reduced the intrinsic tryptophan fluorescence of FtsZ (Y371W) in a concentration dependent fashion. The apparent decrease in the fluorescence values in the presence of varying concentrations of the compound were corrected for the inner-filter effect as described previously (Beuria T K, Santra M K, Panda D. (2005) Sanguinarine blocks cytokinesis in bacteria by inhibiting FtsZ assembly and bundling. Biochemistry. 44, 16584-93). The corrected fluorescence intensities of FtsZ (Y371W) in the presence the compound at different concentrations were used to determine the dissociation constant using a double reciprocal plot (Beuria T K, Santra M K and Panda D. (2005) Sanguinarine blocks cytokinesis in bacteria by inhibiting FtsZ assembly and bundling. Biochemistry. 44,16584-93).

E. coli FtsZ does not contain a tryptophan residue; therefore, a mutant of FtsZ (Y371W) was used, where a tyrosine residue at position 371 has been replaced by a tryptophan residue to study the interaction between the compound and FtsZ. The assembly kinetics of the native and the mutant FtsZ were found to be similar. The mutation did not alter biochemical, functional or physical properties of the native protein. (Beuria T K, Santra M K and Panda D. (2005) Sanguinarine blocks cytokinesis in bacteria by inhibiting FtsZ assembly and bundling. Biochemistry. 44, 16584-93). The compound reduced the tryptophan fluorescence of mutated FtsZ (Y371W) in a concentration-dependent manner (FIG. 5A). The change in the fluorescence intensity was used to calculate the dissociation constant of FtsZ-compound complex. A dissociation constant of 15±1.5 μM was determined using a double reciprocal plot of the binding data (FIG. 5B).

### Example 8

### Circular Dichroism (CD) Measurements

FtsZ (1 μM) was incubated with different concentrations of the compound (0-50 μM) at 25° C. The far-UV CD spectra were recorded using a JASCO spectropolarimeter. The secondary structure was monitored over the wavelength range of 200-260 nm using a 0.1-cm path length cuvette and the ellipticity was determined at 220 nm. Each spectrum was recorded using an average of 3 scans.

The far-UV CD spectra of FtsZ were found to be similar in the absence and presence of the compound indicating that the compound did not change the secondary structure of FtsZ (FIG. 6).

### Example 9

### Determination of Minimum Inhibitory Concentration (MIC) and IC₅₀

The inhibitory effect of the compound on the bacterial division was determined by measuring the absorbance at 600 nm (A₆₀₀). An overnight culture of Bacillus subtilis 168 was diluted several folds (A₆₀₀, 0.1) and grown in the presence of different concentrations of the compound for another 90 min at 37° C. A₆₀₀ was measured at the end of 90 min. Minimum inhibitory concentration (MIC) was calculated as the minimum amount of the compound required to prevent the growth of bacteria in the liquid culture completely and the IC₅₀ was calculated as the concentration of the compound required to inhibit growth by 50% in a liquid culture.

The growth of Bacillus subtilis was found to decrease in the presence of compound in a concentration dependent manner. The concentration that inhibited 50% bacterial growth (IC₅₀) was calculated to be 1.1 μM. A concentration of compound that inhibited bacterial growth visibly (MIC) was calculated to be 3 μM (FIG. 7). The experiment was repeated 5 times.

### Example 10

Differential Interference Contrast (DIC) and Fluorescence Microscopic analysis

An overnight culture of Bacillus subtilis 168 was diluted several folds (A₆₀₀˜0.1) and the diluted culture was grown in the absence or presence of different concentrations of the compound for another 90 min at 37° C. Bacillus subtilis 168 cells were fixed with 0.04% glutaraldehyde plus 2.5% formaldehyde, harvested, and resuspended in LB medium containing 0.25% of agarose. A total of 5 μL of the suspension was placed on a cover slip, and morphology of the bacterial cells was observed under light microscope. FM 4-64 was used to visualize the inner membrane of the bacteria (Beuria T K, Santra M K and Panda D. (2005) Sanguinarine blocks cytokinesis in bacteria by inhibiting FtsZ assembly and bundling. Biochemistry. 44,16584-93; Fujita M, Losick R. (2005) Evidence that entry into sporulation in Bacillus subtilis is governed by a gradual increase in the level and activity of the master regulator Spo0A. Genes and Development. 19, 2236-44). FM 4-64 was added to a growing B. subtilis culture to a final concentration of 1-1.5 μM. After 15 min, cells were observed using a fluorescence microscope (Nikon ECLIPSE TE2000-U) with a 60×objective. The images were captured using a CoolSNAP-Pro camera, and the length of bacterial cells was measured by using IMAGEPRO PLUS software (Media Cybernetics, Silver Spring, Md.).

The compound affected the morphology of the bacteria. It was found that the compound inhibited the growth of Bacillus subtilis 168 and induced filamentation in Bacillus subtilis (FIG. 8A). The average length of Bacillus subtilis cells in the absence and presence of 1 μM concentration of the compound was found to be 4.4±1.3 and 12.4±5.2 μm, respectively (FIG. 8B and Table 1). Further, no bacterium had a length equal to or more than ˜10 μm in the absence of compound whereas 38.5% of the bacteria were having length >10 μm in the presence of 1 μM of the compound. The Differential Interference Contrast (DIC) images and membrane staining with FM 4-64 showed that the compound did not perturb the outer and inner membrane of Bacillus subtilis (FIG. 9). The DIC images showed the presence of constrictions at the division site and FM 4-64 staining showed the formation of septa at the division site in the presence of the compound. However, the formation of filamentous cells in the presence of 1 μM concentration suggested that the compound inhibited cell proliferation by preventing cytokinesis. The data indicated that the septum could form in the presence of compound, but these septa were not functional enough to regulate the cell division.

FM4-64 stained multiple sites where the septa are likely to be formed indicating that FtsZ was able to polymerize in the compound treated cells. The formation of the septa at more than one sites also suggested that the septa were inoperative in the presence of compound.

### Example 11

### Effect of the Compound on the Z-Ring

The effects of the compound on the Z-rings and nucleoids of Bacillus subtilis 168 cells were examined as described recently (Beuria T K, Santra M K and Panda D. (2005) Sanguinarine blocks cytokinesis in bacteria by inhibiting FtsZ assembly and bundling. Biochemistry. 44, 16584-93). Bacillus subtilis 168 (A₆₀₀˜0.3-0.4) cells were incubated in absence and presence of 1 μM concentration of the compound for 90 min. Then, the cells were fixed with 2.5% formaldehyde and 0.04% glutaraldehyde (Beuria T K, Santra M K and Panda D. (2005) Sanguinarine blocks cytokinesis in bacteria by inhibiting FtsZ assembly and bundling. Biochemistry. 44, 16584-93). FtsZ was stained with a polyclonal anti-FtsZ rabbit antibody (Bangalore Genei, India) followed by a Cy3-conjugated goat anti-rabbit secondary antibody (Sigma). Nucleoids were visualized by treating the cells with 0.5 μg/mL DAPI. The compound did not display any background fluorescence. Cells were observed using a fluorescence microscope (Nikon ECLIPSE TE2000-U) with a 60×objective. The images were captured using a CoolSNAP-Pro camera, and the length of a bacterial cell was measured using IMAGEPRO PLUS software (Media Cybernetics, Silver Spring, Md.).

The effects of the compound on the cytokinetic Z-rings of Bacillus subtilis 168 were examined by staining the Z-ring with FtsZ antibody. A typical Z-ring was observed in most of the vehicle-treated (control) Bacillus subtilis cells whereas no multiple Z-rings were observed in the bacterial cells treated with 1 μM concentration of the compound and the cells got elongated. For example, 62% of the control cells contained a one Z-ring whereas none of the cells treated with the compound contained only a single Z-ring (FIG. 10 and Table 1). Again, the compound treated cell displayed a patch of FtsZ in between and above the nucleoids.

Nucleoids in Bacillus subtilis 168 cells were stained using DAPI. The nucleoid segregation was found to be similar in both the treated and untreated cells. The compound treated cells had no detectable effect on nucleoid segregation of Bacillus subtilis. As the bacteria were unable to divide in the presence of the compound, the number of nucleoids in the drug treated cells was found to be higher than that of the control cells. For example, average nucleoids per cell were 2.32 and 8 in the absence and presence of 1 μM of compound 1 (FIG. 10 and Table 1). Further, the frequency of nucleoid per μm of cell length was found to be unaltered in the absence (0.58) and presence (0.65) of 1 μM of compound 1 suggesting that the nucleoid segregation was not perturbed in the treated cells.

DAPI staining showed that the number of nucleoids per unit cell length was not perturbed in the compound treated Bacillus subtilis cells suggesting that the compound did not alter nucleoid segregation in bacteria. Immunostaining of the Z-ring showed the presence of a typical Z-ring in the control cells. However, multiple Z-rings were observed in the compound treated cells and also a few patches of FtsZ were observed in between and over the nucleoids. The findings indicated that FtsZ was able to assemble at the place for the Z-ring formation but was unable to form a functional Z-ring that could proceed to divide the cells normally. Further, the presence of the FtsZ patches over the nucleoid suggests that in the presence of the compound, FtsZ polymers were stabilized that prevented the formation of the Z-ring over the nucleoids.

### Example 12

### The Effects of the Compound on Mammalian Cell Proliferation

The compound inhibited proliferation of HeLa cells in a concentration dependent manner with an IC₅₀ of ˜8 μM. For example, 36% and 64% inhibition of HeLa cell proliferation occurred in the presence of 5 and 10 μM concentration of the compound, respectively. In contrast, 50% inhibition of Bacillus subtilis proliferation occurred in the presence of 1.1 μM of compound suggesting that the compound has much weaker inhibitory effect on mammalian cells as compared to the bacterial cells.

The evidence presented in the present invention strongly suggests that the compound (3-{5-[4-Oxo-2-thioxo-3-(3-trifluoromethyl-phenyl)-thiazolidin-5-ylidenemethyl]-furan-2-yl}-benzoic acid) can directly stabilize the protofilaments; however, it can also stabilize the FtsZ protofilaments in the Z-ring by inhibiting the binding of the negative regulators to FtsZ.

The results suggest that Z-ring dynamicity is important for the bacterial division and it can be targeted for the development of new antibacterial agents.

TABLE 1
Effect of the compound on Z-ring
and nucleoids of Bacillus subtilis^{a} 168

| Description | Control | Compound |
|---|---|---|
| Cell length | 4.4 ± 1.3 μm | 12.4 ± 5.2 μm |
| Percent of cells having Z-ring | 62% | NIL |
| Average Z-ring per cell | 0.63 | NIL |
| Frequency of nucleoids per μm of | 0.58 | 0.65 |
| cell length | | |
| Number of nucleoids per Z-ring | 2.4 | ND |
| Percent of cells having single nucleoids | 10% | NIL |
| Percent of cells having two nucleoids | 71% | 5% |
| Percent of cells having two | 90% | NIL |
| nucleoids having a Z-ring | | |
| Percent of cells having four nucleoids | 19% | 11% |
| Percent of cells having eight | NIL | 84% |
| or more nucleoids | | |

^{a}A minimum of 200 cells were scored for both the cases

1. A pharmaceutical composition comprising an effective amount of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof and pharmaceutically acceptable excipients, wherein the composition has antibacterial activity.

2. The pharmaceutical composition as claimed in claim 1, wherein the effective amount of said 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof is in the range of 1 μM to 5 μM.

3. The pharmaceutical composition as claimed in claim 1, wherein the composition optionally comprises supplementary active compounds selected from a group consisting of antibiotic, antiprotozoal agent, antifungal agent, antipathogen, and antiproliferative agent.

4. The pharmaceutical composition as claimed in claim 1, wherein the composition is in the form of a tablet, capsule, solution or powder.

5. The pharmaceutical composition as claimed in claim 1, wherein the mode of administration of the composition is intravenous, intramuscular or oral.

6. A method of treatment of bacterial infections and associated diseases, wherein said method comprises administering a composition comprising an effective amount of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof and pharmaceutically acceptable excipients.

7. The method as claimed in claim 6, wherein said effective amount is in the range of 1 μM to 5 μM.

8. Use of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof for preparation of a medicament useful for treatment of bacterial infections and associated diseases.

9. Use of 3-{5-[4-Oxo-2-thioxo-3-(3-trifluoromethyl-phenyl)-thiazolidin-5-ylidenemethyl]-furan-2-yl}-benzoic acid or pharmaceutically acceptable salt thereof in an effective amount ranging from 1 μM to 5 μM for preparation of a medicament useful for treatment of bacterial infections and associated diseases.
